(19) United States
(12) Patent Application Publication
(10) Pub. No.: US 2005/0020921 A1
(43) Pub. Date: Jan. 27, 2005

(54) Ultrasonic sensor garment for breast tumor

(75) Inventors: Richard Glassell, Knoxville, TN (US); Barry L. Burks, Oak Ridge, TN (US); Diedre D. Falter, Knoxville, TN (US)

Correspondence Address: PITTS AND BRITTIAN P C, P O BOX 51295, KNOXVILLE, TN, 37950-1295, US

(73) Assignee: TPG Applied Technology, Knoxville, TN (US)

(21) Appl. No.: 10/616,559

(22) Filed: Jul. 10, 2003

(51) Int. Cl. A61B 8/14

(52) U.S. Cl. 600/463

(57) ABSTRACT

A cancer detection system having a plurality of ultrasonic sensors positioned about a garment worn over at least one breast. The sensors transmit a signal that is received by the other sensors. A processor records the amplitude and time-of-flight of the received signals. The signals include both direct line-of-flight signals and reflected signals. In one embodiment, the processor performs tissue structure analysis. In another embodiment, the recorded data is sent to a remote processor for long term storage, tissue structure analysis, and/or addition to a chronological profile.

### CROSS-REFERENCE TO RELATED APPLICATIONS

Not Applicable

### STATEMENT REGARDING FEDERALLY SPONSORED RESEARCH OR DEVELOPMENT

Not Applicable

### BACKGROUND OF THE INVENTION

1. Field of Invention

This invention pertains to a system for detecting cancerous tumors within a human breast. More particularly, this invention pertains to a system for ultrasonically monitoring and logging tissue development within human breasts in order to detect localized tissue abnormalities.

2. Description of the Related Art

Breast cancer claims the lives of tens of thousands of women every year. Many of these victims could have survived if the cancer had been detected and treated in its primary stages. The most effective method of detecting this disease in its primary stages is regular periodic breast examinations. Currently, the three most common methods of breast examination are monthly self-examinations, annual mammograms, and clinical examinations. Monthly self-examinations require a woman to detect by touch and identify an abnormal “lump” within her breast using her hands. This method of palpation is limited in that by the time a “lump” is large enough to be felt by the woman, abnormal tissue development has progressed past its primary stages. Additionally, certain populations of women have naturally “lumpy” breast tissue. This condition introduces an additional degree of difficulty for a woman attempting to detect an abnormal “lump”.

Annual mammograms are currently the standard in breast examinations. These examinations include compressing a breast and passing X-rays through the breast in order to produce an image of the entire organ. Mammograms are limited in that they are inconvenient, somewhat painful, use radiation, and produce only a “snapshot” of the organ. Further, a shortage in radiologists has presented additional limitations to this method. However, the most significant limitation associated with this method of breast cancer detection is the significant percentage of cancerous tumors left undetected.

Clinical examinations, like mammograms, are important in detecting breast cancer. However, also like mammograms, clinical examinations are inconvenient and provide only a “snapshot” of a breast.

Ultrasonic imaging is a useful tool for detecting abnormal tissue development within a female breast. Recent studies have revealed that abnormal tissue development missed by mammograms is detectable with ultrasonic technology. However, ultrasonic imaging is highly dependent on operator technique and is a very tedious procedure that can possibly result in an incomplete scan. Further, clinical ultrasounds require the application of messy ultrasound gels for eliminating an interfering layer of air between the sensor and the patient.

The apparatus of U.S. Pat. No. 6,117,080 issued to Schwartz is a system utilizing ultrasonic energy for detecting breast cancer. More specifically, the apparatus transmits ultrasonic energy and reads the corresponding echoes produced by a patient's tissue to determine the presence of a tumor. This system is limited in that it requires the sliding of a scanning head across the patient's breast by a trained ultrasonographer, which consequently requires a clinical visit. Further, a waterbag device is required as a coupling agent for the scanning head and the patient's breast in order for the apparatus to reveal a clear and accurate image.

The apparatus of U.S. Pat. No. 5,997,477 issued to Sehgal also utilizes ultrasonic energy for detecting breast tumors. This apparatus employs a driving signal transmitter that directs a first signal toward a calcification that causes the calcification to resonate. The apparatus further employs an imaging signal transmitter that directs a second signal toward the calcification. A receiver then detects a resonance echo signal produced by the first signal and second signal in order to determine characteristics of the calcification under consideration. This apparatus is limited in that it requires a plurality of types of transmitters along with corresponding receivers in order to detect tumors within a human breast.

Finally, the apparatus of U.S. Pat. No. 5,678,565 issued to Sarvazyan is a system for utilizing ultrasonic energy combined with a pressure sensing device for detecting tumors within a human breast. A scanning head containing a pressure sensor and ultrasonic scanning capabilities is slid across a breast so that the pressure sensor detects tissue elasticity changes while the ultrasonic component processes backscattered ultrasonic signals. The combination of readings reveals the presence of a cancerous tumor. However, this apparatus is limited in that it requires both pressure and ultrasound readings in order to detect a cancerous tumor. Further, the reliable use of this apparatus requires a trained ultrasonographer, which requires a clinical visit.

### BRIEF SUMMARY OF THE INVENTION

In accordance with the present invention there is provided a cancer detection system that utilizes ultrasonic technology for gaining information regarding the tissue development of a female breast. The cancer detection system includes a plurality of ultrasonic sensors that are held in position around a patient's breasts by a garment. The sensors, in one embodiment, are transceivers and, in another embodiment, are individual transmitters and receivers. A transmitting sensor emits an ultrasonic pulse that is received by the receiving sensors that have a direct line-of-flight to the transmitting sensor. The time-of-flight of the received signal indicates the distance between the transmitting sensor and the receiving sensor. Density changes in the breast tissue, which may be indicative of a tumor or be due to a normal feature of the breast, affect the amplitude of the received signal. In another embodiment, the cancer detection system records reflected signals in addition to the direct signals, thereby increasing the resolution and precision of the cancer detection system.

A multitude of line-of-flight data collected from all sensors with all sensors sequentially serving as a transmitting sensor is processed to produce a pair of virtual breasts. The virtual breasts are collected over a period of time and are compared one to another to determine if any changes are occurring in the breast, other than natural changes resulting from normal physiological changes of the breast tissue.

In one embodiment, the cancer detection system is used within the home and communicates with the doctor of the patient by way of the Internet. In another embodiment, the cancer detection system is used in a clinical setting. The cancer detection system stores all information from the periodic examinations of a particular patient and builds a chronological profile of her breast tissue development. If a localized tissue abnormality becomes apparent, proper action is taken to determine if the abnormality is the early development of a cancerous tumor. Because the cancer detection system detects abnormal tissue development in its primary stages, if a cancerous tumor is found, an immediate treatment will greatly increase the probability of a successful treatment.

The cancer detection system, in one embodiment, includes a local processing device that loads a breast examination program from a remote processing device by way of the Internet. The local processing device utilizes a sensor garment, comprised of a number of ultrasonic transceivers, to produce an ultrasonic image of the tissue of a breast. The ultrasonic transceivers are positioned about the sensor garment such that they surround an entire breast. Once positioned around a breast, the ultrasonic transceivers transmit and receive a series of signals that are analyzed in the amplitude and time domain in order to detect a localized tissue abnormality and to isolate the location of the potentially cancerous abnormality within the breast. The positioning and operation of the ultrasonic transceivers allow a woman to obtain a breast examination without the assistance of a trained clinician. The ultrasonic images acquired from an examination are stored in the local processing device until they are loaded to the remote processing device. From the remote processing device, a doctor examines the results of the recent examination with respect to the results of previous examinations. These comparisons reveal, if present, the development of a localized tissue abnormality. The early detection of these tissue abnormalities allows doctors to diagnose and treat the abnormalities in order to prevent the development of a fatal cancerous tumor.

### BRIEF DESCRIPTION OF THE SEVERAL VIEWS OF THE DRAWINGS

The above-mentioned features of the invention will become more clearly understood from the following detailed description of the invention read together with the drawings in which:

FIG. 1 is a pictorial block diagram of one embodiment of a cancer detection system;

FIG. 2 is block diagram illustrating one embodiment of the electrical components of the cancer detection system of FIG. 1;

FIG. 3 is a flow diagram illustrating the operation of one embodiment of a local processing device;

FIG. 4 is a perspective view of one embodiment of an ultrasonic device;

FIG. 5 is a side elevation view of the ultrasonic device of FIG. 4 in section;

FIG. 6 is a flow diagram illustrating the transmission and reception of a single ultrasonic signal;

FIG. 7 is a sectional view of a breast accommodating cup illustrating the detection of a localized tissue abnormality by way of direct line-of-flight signal components;

FIG. 7a is a pictorial view of an ultrasonic beam between a transmitting sensor and a receiving sensor with a large obstruction partially in the beam;

FIG. 7b is a pictorial view of an ultrasonic beam between a transmitting sensor and a receiving sensor with a small obstruction;

FIG. 8 is a timing diagram illustrating the signal analysis utilized in determining the presence and location of a localized tissue abnormality;

FIG. 9 is a sectional view of a breast accommodating cup illustrating the detection of a localized tissue abnormality by way of reflected signal components;

FIG. 10 is a sectional view of a breast accommodating cup further illustrating the detection of a localized tissue abnormality by way of direct line-of-flight signal components; and

FIG. 11 is a sectional view of a breast accommodating cup illustrating the signal coverage provided by the present invention.

### DETAILED DESCRIPTION OF THE INVENTION

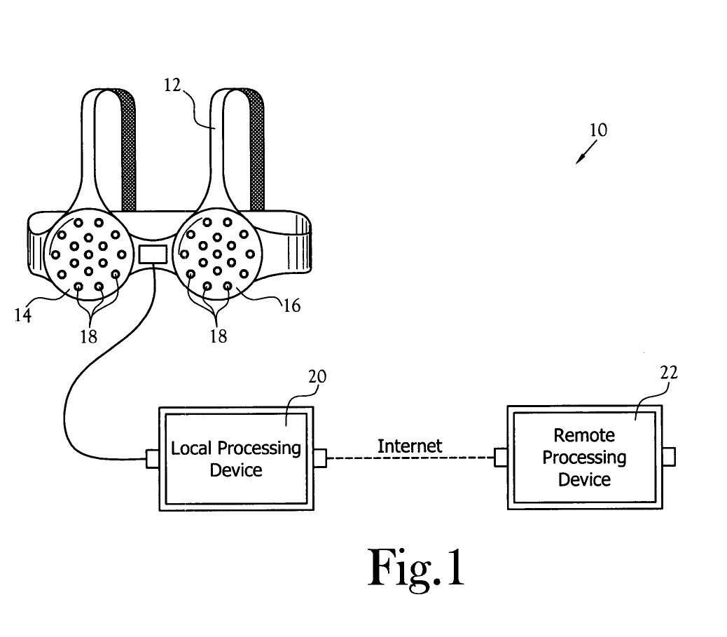

One embodiment of a cancer detection system constructed in accordance with the various features of the present invention is illustrated generally at 10 in FIG. 1. The cancer detection system 10 utilizes ultrasonic technology for gaining information regarding the tissue development of a female breast. The illustrated embodiment of the cancer detection system 10 includes a portion that is used within the home and that communicates with a remote portion by way of the Internet. The cancer detection system 10 stores all information from the periodic examinations of a particular patient and builds a chronological profile of her breast tissue development. If a localized tissue abnormality becomes apparent, proper action is taken to determine if the abnormality is the early development of a cancerous tumor. Because the cancer detection system 10 detects abnormal tissue development in its primary stages, if a cancerous tumor is found, an immediate treatment will greatly increase the probability of a successful treatment.

FIG. 1 illustrates a pictorial block diagram of one embodiment of a cancer detection system 10 that includes a sensor garment 12, a local processing device 20, and a remote processing device 22. Another embodiment includes a sensor garment 12 and a processor, which performs the functions of the local processing device 20 and the remote processing device 22. This embodiment is suitable for a clinical environment or where the processing of the data is to be performed by the data acquisition processor, which in the first embodiment is the local processor 20. Hereinafter, the embodiment illustrated in FIG. 1 is discussed, although the invention is not limited to such an embodiment.

The sensor garment 12, in the illustrated embodiment, is a garment that resembles a sports bra. The sensor garment 12 includes a first cup 14 and second cup 16 for accommodating breasts during an examination. The first cup 14 and the second cup 16 each include a number of sensors 18. In the illustrated embodiment, the sensors 18 are transceivers that transmit and receive ultrasonic energy. In another embodiment, the sensors 18 include both individual transmitters and receivers. The sensors 18 are mounted within the sensor garment 12 such that they completely surround a breast and provide signal coverage for the entire organ. The ultrasonic signals transmitted and received by the sensors, or ultrasonic devices, 18 produce the breast tissue information necessary to detect a localized tissue abnormality. One of the ultrasonic devices 18 transmits a pulse signal, which is received by the other ultrasonic devices 18 that are in a direct line of site of the transmitting ultrasonic device 18. As tissue density changes for regions in the direct line-of-flight between the transmitting sensor 18 and the receiving sensor 18, the signal received by the receiving sensor 18 is altered relative to previously collected/stored data.

The sensor garment 12 fits firmly against the breasts of the patient such that all ultrasonic devices 18 are in solid contact with the breasts. In one embodiment, the devices 18 are fixed to the inside surface of the garment 12 such that one face of the sensor device 18 is in contact with the patient's skin. A firm fitting garment also assists the patient in wearing the garment in the same relative position for each examination such that the cancer detection system 10 reveals consistent results. Because a woman may not wear the sensor garment 12 in exactly the same position for each examination, the computer processing of the cancer detection system 10 references the patient's chronological profile and compensates for the misalignment.

A local processing device 20 in electrical communication with the ultrasonic devices 18 is employed for system control, data collection, user interface, and communication. In one embodiment, prior to each examination, a patient enters a password into the local processing device 20 to identify the patient and provide patient privacy and security. Upon each examination request, a breast examination program is loaded to the local processing device 20 by a remote processing device 22 after the remote processing device 22 confirms the user password. Once programmed, the local processing device 20 governs the breast examination, collects the readings from the ultrasonic devices 18, performs noise reducing signal processing, and temporarily stores this information. Once the readings have been collected by the local processing device 20, the remote processing device 22 retrieves the readings, performs signal analysis, and adds the results to the chronological profile of the patient. Signal processing is then performed on the profile in order to detect any developing localized tissue abnormalities. If alerted to a potential abnormality, the doctor of the patient then accesses the patient's profile from the remote processing device 22 and conducts his/her diagnosis.

In the illustrated embodiment, the local processing device 20 communicates with the remote processing device 22 by way of the Internet. In one embodiment, the Internet-based connection is achieved by connecting the local processing device 20 and the remote processing device 22 to respective general purpose computers capable of accessing the Internet. In another embodiment, the Internet-based connection is achieved by providing the local processing device 20 and the remote processing device 22 with a capability for accessing the Internet. However, those skilled in art will recognize that the utilization of an Internet-based connection is not required to remain within the scope or spirit of the present invention. For example, in one embodiment, the information obtained from the sensor garment 12 and the local processing device 20 is stored on a data storing medium and physically delivered to the patient's doctor. In another embodiment, the local processing device 20 is connected to the remote processing device 22, such as through a network connection.

The remote processing device 22 uses signal analysis algorithms to examine the most recent data and compare that data to previously recorded data. If, during the analysis and comparisons, possible localized tissue changes are detected, then the remote processing device 22 generates an alert. The alert prods a physician to review the data and determine whether additional diagnostic procedures need to be pursued. The remote processing device 22, in various embodiments, 1) communicates with the sensor garment 12 via the local processing device 20 to control data acquisition, data analysis, and data storage; 2) provides the test program for the sensor garment 12; 3) collects and stores transmission data; 4) performs the analysis of current and previously collected data; 5) runs updated software routines against the collected data as the software evolves; and 6) communicates with the patient and physician to provide status of the data analysis and alerts if suspect tissue growth is detected.

In another embodiment, the patient puts on the sensor garment 12 and the local processing device 20 controls the data acquisition for a complete breast examination. The local processing device 20 stores the collected data for transferal to the remote processing device 22, which stores the data and performs processing for diagnoses.

The sensor garment 12 provides the function of positioning the sensors 18 against the breast. The sensor garment 12, in combination with the coupling agent 26 (discussed below), also functions to secure the sensors 18 against the breast. In one embodiment, the local processing device 20 provides the function of acquiring the data received by the sensors 18. The remote processing device 22 provides the function of processing the data acquired by the local processing device 20. In another embodiment, a single processing device performs the functions of acquiring data from the sensors 18 and processing the acquired data.

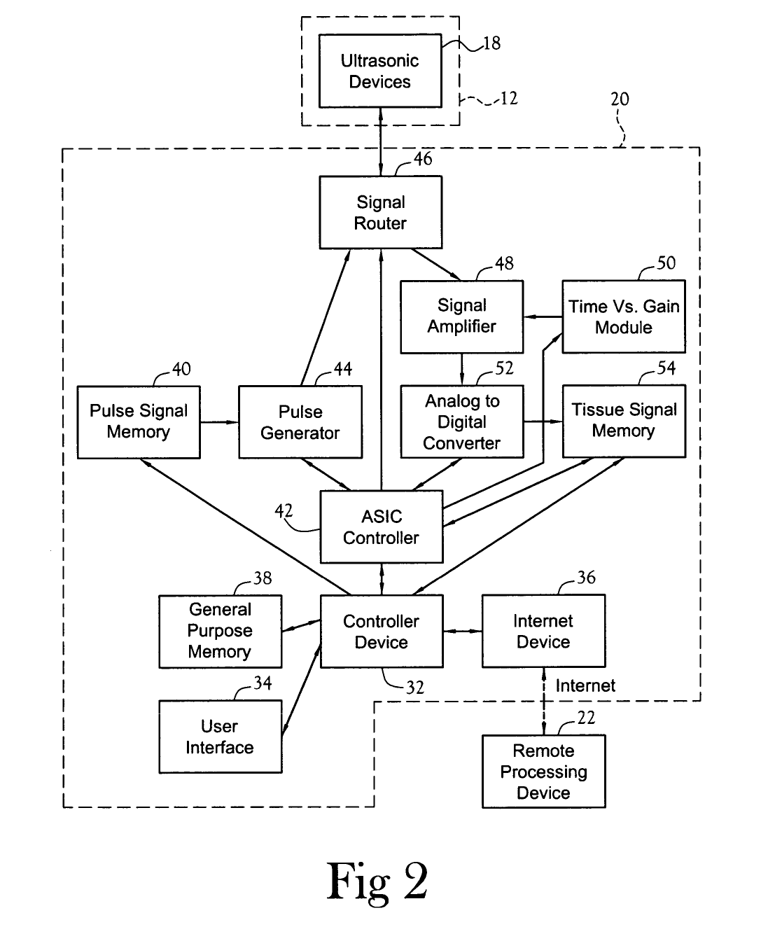

FIG. 2 is a block diagram illustrating one embodiment of the electrical components of the cancer detection system 10. In the illustrated embodiment, a controller device 32 governs the general operation of the local processing device 20. The controller device 32 communicates with the patient through a user interface 34 and upon the patient's request of a breast examination, the controller device 32 obtains the breast examination program from the Internet through an Internet device 36 and stores the program information in a general purpose memory 38. After the breast examination program has been loaded, the corresponding pulse signal to be transmitted through the breast is stored in a pulse signal memory 40.

An application specific integrated circuit (ASIC) controller 42 is employed to conduct the data acquisition. After being prompted by the controller device 32, the ASIC controller 42 activates a pulse generator 44 that reads the specified pulse signal from the pulse signal memory 40, converts the digital signal to an analog signal, and transmits the signal to a signal router 46, which distributes the signal to a specific sensor 18. The signal router 46 then directs the signal received by a specific sensor 18 to a signal amplifier 48. The ASIC controller 42 provides the desired magnitude of signal amplification to a time vs. gain adjustment module 50, which adjusts the signal amplifier 48 accordingly. The amplified signals are then read by an analog-to-digital converter 52, which digitizes each signal. The digitized signals are stored in a tissue signal memory 54 until the controller device 32 requests the signals for noise reducing signal processing, which is performed by the controller device 32. The controller device 32 then transmits the results through the Internet device 36, across the Internet, and to the remote processing device 22.

Those skilled in the art will recognize that electronic configurations for the local processing device 20 other than the previously discussed configuration may be used without interfering with the scope or spirit of the present invention. For example, in another embodiment, the controller device 32 includes programming for performing the tasks of the ASIC controller 42. Due to the high-speed nature of the data collection process, this embodiment requires the controller device 32 to be a high-speed device.

Another embodiment of the local processing device 20 has an analog-to-digital converter (ADC) and a digital-to-analog converter (DAC) for each sensor 18. A processor sends data to one sensor's DAC for that sensor to transmit, all the other sensors receive the transmitted signal and the processor transfers the data acquired from each ADC to memory during the data acquisition phase. Each sensor 18 sequentially transmits a signal for a complete examination. Once all the data is acquired and stored in memory, the data can be processed either with the local processing device 20, in one embodiment, or processed remotely by the remote processing device 22, in another embodiment.

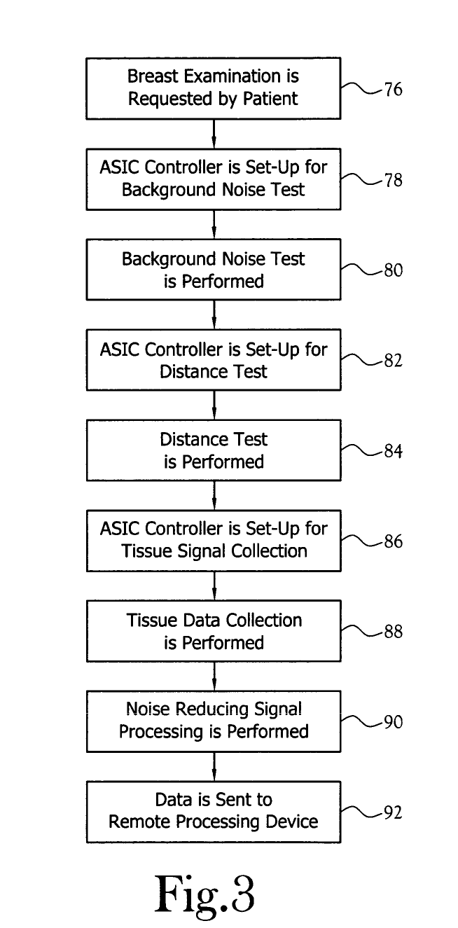

FIG. 3 is a flow diagram illustrating the general operational behavior of the embodiment of the local processing device 20 illustrated in FIG. 2. A breast examination begins at block 76 where a breast examination is requested by a patient by way of the user interface 34. Once requested, a breast examination program is loaded by way of the Internet from the remote processing device 22. Then, at block 78, the sampling parameters of the ASIC controller 42 are set up by the controller device 32 for a background noise test. The background noise test is the acquisition of noise such as the patient's heartbeat, blood flow, ultrasonic sensor component noise, electronics noise, or external noise such as conversation. The background noise test is independently and sequentially performed for each sensor 18 in order to determine the characteristics of the background noise at the location of each sensor 18. The background noise test allows the cancer detection system 10 to account for and eliminate signal degenerating noise during signal processing. The background noise test is performed at block 80.

At block 82, the sampling parameters of the ASIC controller 42 are set up by the controller device 32 for a distance test. The distance test determines the physical size of a breast at the time of examination and provides data used by the processing routines. More specifically, the distance test determines a signal's time-of-flight between any two sensor 18 with a direct line-of-flight in order to calculate the desired initiation and duration of received signal sampling. The time-of-flight for a signal between two sensors 18 is determined by beginning sampling at a receiving ultrasonic device at the moment a pulse is transmitted from a corresponding transmitting ultrasonic device. The number of samples collected before the pulse is detected by the receiving ultrasonic device is converted to a value of time by considering the current sampling rate. This value of time is the time-of-flight for a signal of the corresponding sensor 18 combination. Additionally, once the physical size of a breast has been calculated, the maximum distance possible for a reflected signal to travel before reaching its receiving ultrasonic device is determined and converted to a corresponding maximum propagation time. Therefore, the time-of-flight and the maximum propagation time allow the local processing device 20 to establish an initiation and duration for the sampling of a received signal for each of the sensor 18 combinations.

It can be understood from previous discussion that the results of the distance test are used to calibrate the time vs. gain module 50 of FIG. 2. The distance test is performed at block 84. Finally, the sampling parameters of the ASIC controller 42 are set up by the controller device 32 for tissue data collection at block 86. The tissue data collection, performed at block 88, is illustrated and discussed in subsequent discussion.

In one embodiment, as a form of noise reducing signal processing, a packet of 1000 signals is transferred for each ultrasonic device 18 transmitter-receiver combination. The 1000 values received are then averaged to eliminate any random noise. The noise reducing signal processing is performed at block 90 by the controller device 32. The averaged signal value is then stored in the general purpose memory 38 until the complete set of data from the tissue data collection is transferred to the remote processing device 22 at block 92.

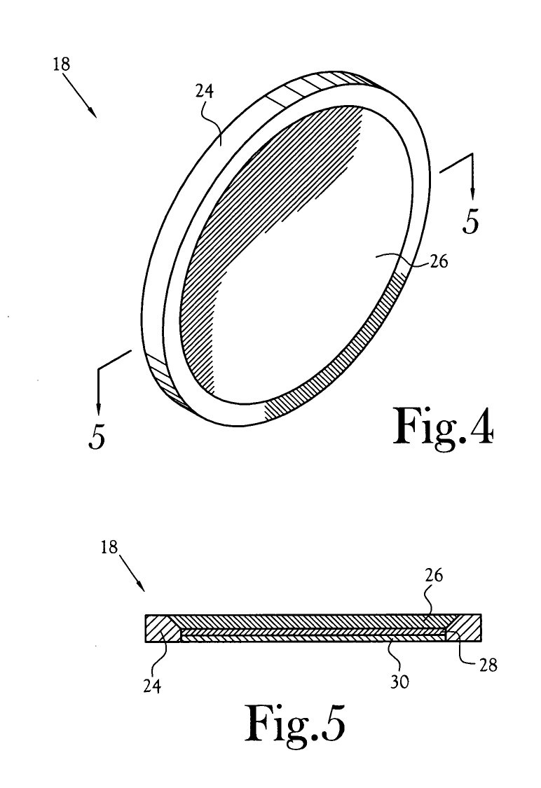

FIG. 4 illustrates a perspective view of a sensor device 18 of FIG. 1, and FIG. 5 illustrates the ultrasonic device 18 in section, taken along lines 5-5. In one embodiment the sensor device 18 is an ultrasonic transducer. In another embodiment, the ultrasonic transducer is a polyvinylidend fluoride (PVDF) piezoelectric transducer. In still another embodiment, the ultrasonic transducer is a ceramic piezoelectric transducer.

The ultrasonic device 18 is a coin-shaped device, which, in one embodiment, is 0.25 inches in diameter and 0.25 inches in depth. Those skilled in the art will recognize that other shapes and dimensions for the ultrasonic device 18 may be used without interfering with the scope or spirit of the present invention. The sensor device 18 of the illustrated embodiment includes a housing 24 for mounting the sensor 18 within the sensor garment 12 and for accommodating a coupling agent 26, a transceiver 28, and a transceiver backing 30. The coupling agent 26 provides connectivity between the transceiver 28 and the breast and eliminates an air boundary layer between the transceiver 28 and the breast. The coupling agent 26 is composed of a material that allows ultrasonic energy to pass through the coupling agent 26 in the same manner that ultrasonic energy passes through human tissue. The characteristics of the coupling agent 26 eliminate the necessity of the messy ultrasound gel required in prior art clinical examinations. The coupling agent 26 of the illustrated embodiment has a contour that is a truncated cone, defined by the housing 24 and the transceiver 28, in order to guide ultrasonic energy transmitted and received by the transceiver 28. The transceiver 28 of the illustrated embodiment is a piezoelectric transceiver that is capable of transmitting and receiving ultrasonic energy. Those skilled in the art will recognize that other devices may be used without interfering with the scope or spirit of the present invention.

The transceiver backing 30 and the housing 24 are constructed of a material that absorbs ultrasonic energy such that the signal emitted from the transceiver 28 is focused in the direction of the coupling agent 26 and the signal received by the sensor 18 is focused toward the transceiver 28.

The ultrasonic signal transmitted by the sensor 18 is an ultrasonic pulse that propagates through a breast. With a point transmitting source, a signal radiates with an expanding spherical pattern. Considering the structure of the sensor 18 depicted in FIG. 4, in the embodiment in which the sensor 18 is 0.25 inch in diameter, the transmitting surface 28 of the sensor 18 is slightly smaller than the 0.25 inch diameter of the complete device. The radiation pattern of the ultrasonic sensor 18 is spherical, but with a base diameter of slightly less than 0.25 inches.

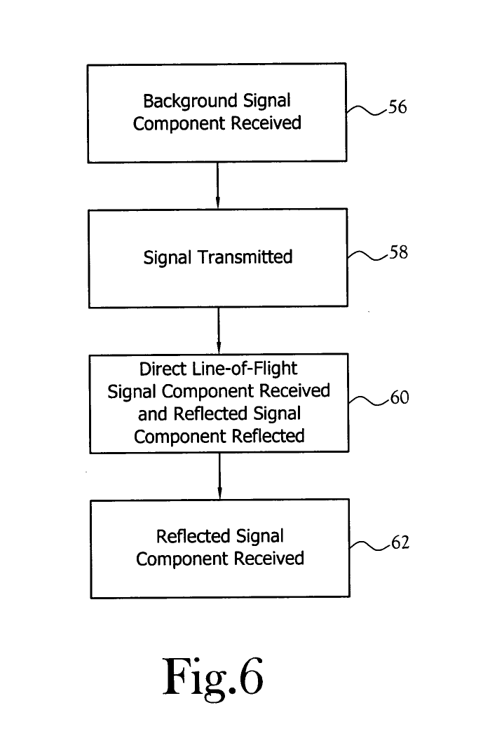

FIG. 6 is a flow diagram illustrating a single signal transmission and reception as performed by the ultrasonic devices 18. A receiving ultrasonic device receives a signal having three signal components, namely a background signal component, a direct line-of-flight signal component, and a reflected signal component. The background signal component is acquired by the receiving ultrasonic device 18 prior to the reception of a signal from a transmitting ultrasonic device at block 56. The background signal component comprises the background noise detected by the receiving sensor 18, such as the patient's heartbeat, blood flow, ultrasonic sensor component noise, electronics noise, and external noise such as conversation. The background signal component allows the cancer detection system 10 to account for and eliminate signal degenerating noise during signal processing.

At block 58, the transmitting sensor 18 transmits a pulsed signal that propagates through breast tissue. Although only one signal is transmitted, the receiving sensor 18 receives the transmitted signal as the direct line-of-flight signal component and the reflected signal component. The direct line-of-flight signal component is the portion of the signal that has a direct line-of-flight from a transmitting sensor 18 to a receiving sensor 18. The function of the direct line-of-flight signal component is to detect a localized tissue density change (normal or abnormal) through the analysis of the signal component's amplitude at a receiving sensor 18 and to provide the cancer detection system 10 with a location of the localized tissue density change. Because the direct line-of-flight signal component travels a lesser distance than the reflected signal component, the direct line-of-flight signal component will arrive at the receiving sensor 18 prior to the reflected signal component, as indicated at block 60.

The reflected signal component is the portion of a signal that reaches the receiving sensor 18 after reflecting off of a localized tissue abnormality. The function of the reflected signal component is to detect and determine the size of a localized tissue abnormality through analysis of the time-of-flight of the signal component, as revealed in subsequent discussion. The reflected signal component is received by the receiving ultrasonic device 18 at block 62.

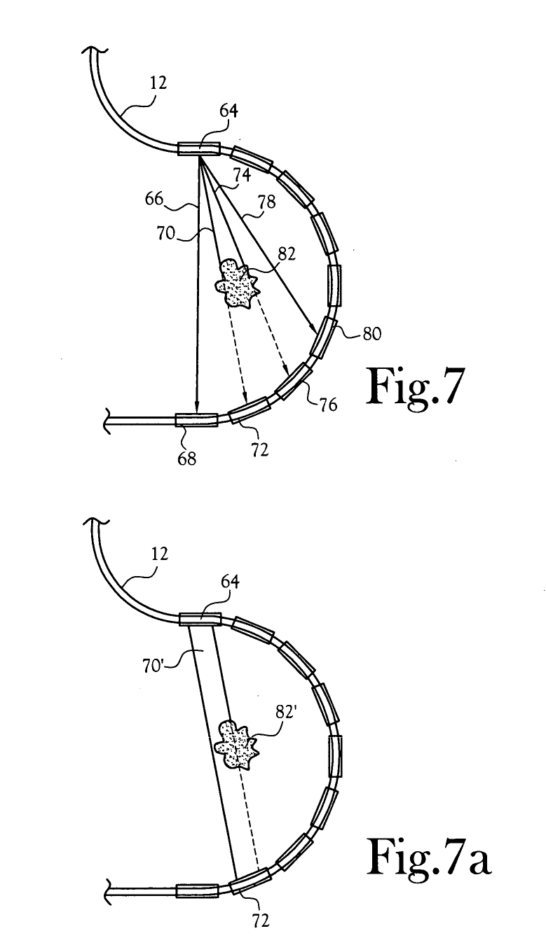

FIG. 7 is a sectional view of the sensor garment 12 illustrating the detection of a localized tissue abnormality by direct line-of-flight signal components. In the illustration, a transmitting ultrasonic device 64 emits a signal that propagates through the breast tissue; however, FIG. 7 depicts only the direct line-of-flight signal components for a few receiving sensors 68, 72, 76, 80. More specifically, a first direct line-of-flight signal component 66 is received by a first receiving ultrasonic device 68, a second direct line-of-flight signal component 70 is received by a second receiving ultrasonic device 72, a third direct line-of-flight signal component 74 is received by a third receiving ultrasonic device 76, and a fourth direct line-of-flight signal component 78 is received by a fourth receiving ultrasonic device 80. In one embodiment, a single signal is transmitted and the receiving sensors 68, 72, 76, 80 simultaneously monitor for received signals. In another embodiment, the sensors 68, 72, 76, 80 sequentially monitor for a series of signals transmitted by the transmitted sensor 64.

The first direct line-of-flight signal component 66 and the fourth direct line-of-flight signal component 78 reach their respective receiving ultrasonic devices without encountering a localized tissue abnormality. However, the second direct line-of-flight signal component 70 and the third direct line-of-flight signal component 74 encounter a localized tissue abnormality 82, which is a region, or volume, of tissue having different density than the surrounding region, before reaching their respective receiving ultrasonic devices. The effect of a localized tissue abnormality 82 is to reduce the amplitude of the signal received by sensors 72 and 76.

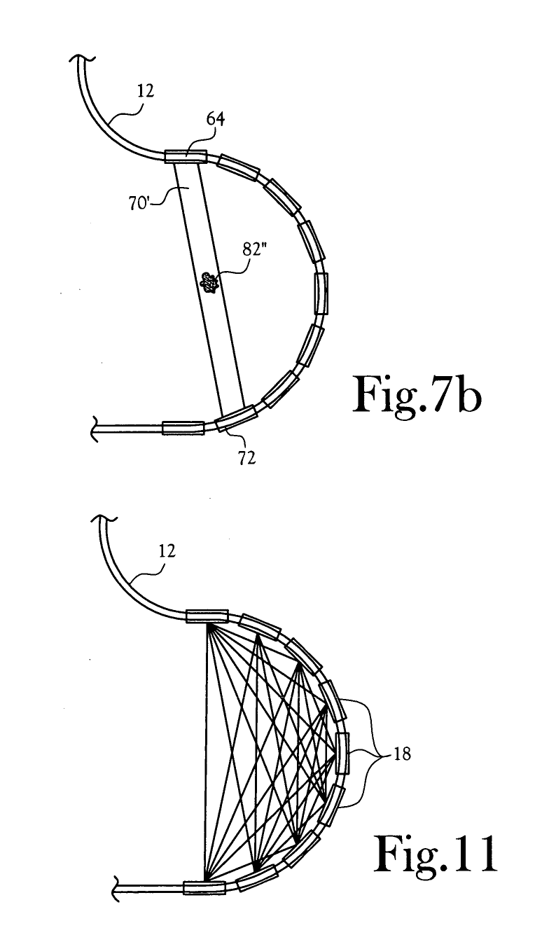

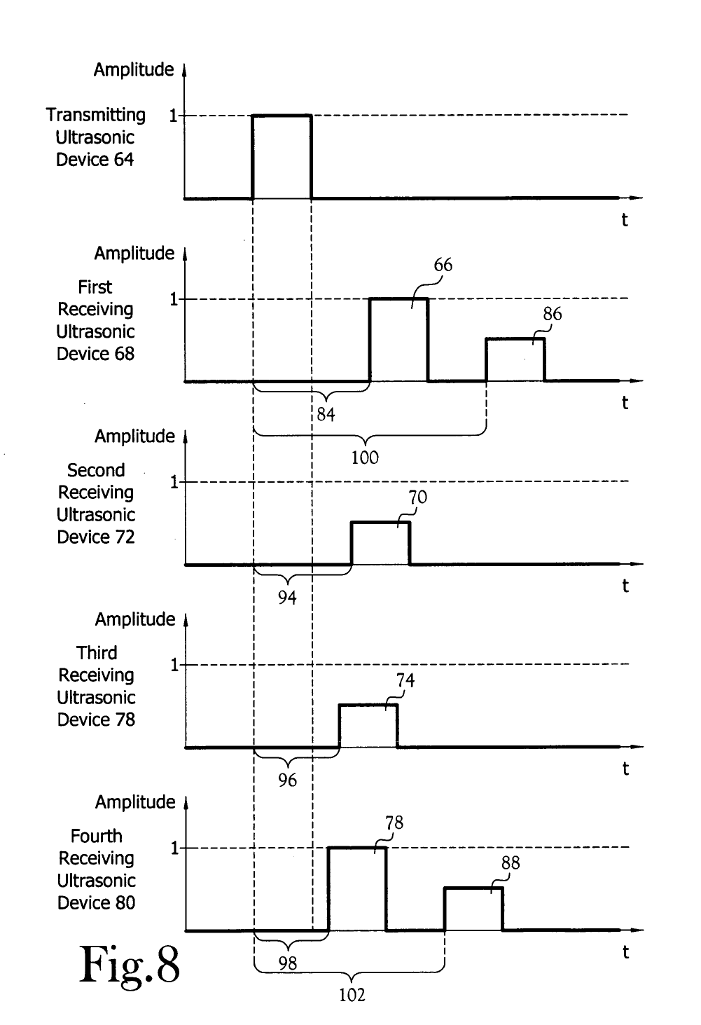

FIG. 8 is a timing diagram that illustrates the direct line-of-flight signal components depicted in FIG. 7. FIG. 8 illustrates the embodiment in which a single pulse is transmitted and the receivers simultaneously monitor. The top diagram shows the transmitted signal emitted by transmitting ultrasonic device 64. The diagrams below illustrate the corresponding signal components received by the receiving ultrasonic devices 68, 72, 76, 80.

FIG. 8 illustrates a first time delay 84 that was previously calculated by the distance test as the time-of-flight for a signal traveling between the transmitting ultrasonic device 64 and the first receiving ultrasonic device 68. This time delay 84 corresponds to the distance between the transmitting sensor 64 and the receiving sensor 68. The speed of sound in fat tissue has been determined to be 0.145 centimeters per microsecond. By multiplying the time 84 in microseconds by this factor, the number of centimeters between the sensors 64 and 68 is determined. In one embodiment, the first receiving ultrasonic device 68 does not begin sampling the first direct line-of-flight signal component 66 until after the calculated time delay 84. Also, as calculated during the distance test, the first receiving ultrasonic device 68 does not discontinue sampling the first direct line-of-flight signal component 66 until after the expiration of the maximum propagation time. In the same manner, a second time delay 94, a third time delay 96, and a fourth time delay 98 dictate the distance between the sensors and the sampling initiation of the second receiving ultrasonic device 72, the third receiving ultrasonic device 76, and the fourth receiving ultrasonic device 80, respectively. The duration of sampling for each receiving ultrasonic device is controlled by its corresponding maximum propagation time.

Ultrasonic signals are attenuated by fat tissue. The amount of attenuation is determined by the distance that the signal travels through fat. For any non-fat tissue that the signal passes through, for example, a localized tissue abnormality, the attenuation will vary. A normalized amplitude can be determined by calculating the expected amplitude of the signal for the distance that the signal travels through fat tissue, which is known, as described above. An amplitude less than this normalized value indicates an area of denser tissue in the signal path.

The first direct line-of-flight signal component 66 does not encounter a localized tissue abnormality, and it has a normalized amplitude value of 1 at the time it is received. The same is true for the fourth direct line-of-flight signal component 78. However, the second and third direct line-of-flight signal components 70 and 74 do encounter a localized tissue abnormality, and they have a normalized amplitude value of less than 1 at the time they are received. These levels are indicated on FIG. 8. From this information, it can be concluded that there is an obstruction in the signal paths 70 and 74 that possibly indicate a localized tissue abnormality 82. In one embodiment, a threshold value can be applied to the normalized value to indicate that the abnormality 82 is of such a size or density that it obstructs the signal a specified amount. In another embodiment, the normalized amplitude is used to determine the size of the abnormality 82. That is, larger abnormalities attenuate more.

FIGS. 7a and 7b illustrate the signal path between the transmitting sensor 64 and the receiving sensor 72 for two sizes of localized tissue abnormalities 82′ and 82″. The sensors 18 have a circular transducer, which in one embodiment is less than 0.25 inches. Accordingly, a transmitter and a receiver directly opposite each other have a signal path that is cylindrical in shape and extends between the transmitter and receiver. For receivers that are not directly opposite the transmitter, the signal path becomes an oblique cylinder, with the cylinder becoming more oblique as the receiving sensor deviates further from the perpendicular of the transmitting sensor.

FIG. 7a illustrates a localized tissue abnormality 82′ that is large, but does not fall totally within the signal path 70′. FIG. 7b illustrates a localized tissue abnormality 82″ that is smaller, but does fall within the signal path 70″. In each of these instances, the amplitude of the signal received by the sensor 72 will be reduced. The received signal 70′ and 70″ does not indicate where along the signal path the abnormalities 82′ and 82″ are located.

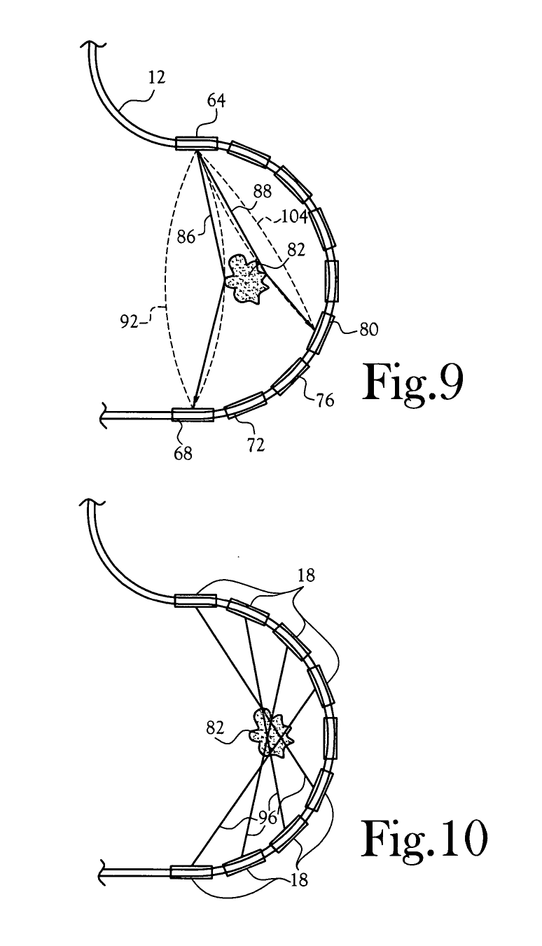

FIG. 9 is a sectional view of the sensor garment 12 illustrating the reflected signal components for the transmitted signal illustrated in FIG. 7. A first reflected signal component 86 is reflected by the localized tissue abnormality 82 and is received by the first receiving ultrasonic device 68. Because the first reflected signal component 86 travels a greater distance and is reflected by the localized tissue abnormality 86, the first reflected signal component 86 is received after the first direct line-of-flight signal component 66 and has a lesser amplitude value. FIG. 8 illustrates the time-of-flight 100 for the received signal 86. This time-of-flight 100 determines the distance that the signal 86 traveled. The distance traveled by a reflected signal component is the sum of the distance traveled prior to encountering a localized tissue abnormality 82 and the distance traveled after encountering the localized tissue abnormality 82. At the point where a reflected signal component encounters a localized tissue abnormality, the propagation path of the signal is altered. A geometric surface is calculated such that any possible location of a propagation path alteration associated with a given total distance traveled by a reflected signal component 86 is located on the geometric surface. Therefore, knowledge of the distance covered by a reflected signal component 86 allows the location of a surface of the localized tissue abnormality 82 to be determined within a geometrically calculated three-dimensional surface 92 that has a longitudinal axis corresponding to the direct line-of-flight signal component. The degree of location provided by the reflected signal components complements, confirms, and refines the locations provided by the direct line-of-flight signal components, as subsequently illustrated.

Similarly, the characteristics of a fourth reflected signal component 88, received by the fourth receiving ultrasonic device 80, are illustrated in FIG. 8 and FIG. 9. The characteristics of the received signal, including a second reflected signal component time-of-flight 102, are analyzed in the way the characteristics of the first reflected signal component 86 are analyzed. Therefore, a corresponding geometrically calculated three-dimensional surface 104 is utilized to determine the location of a surface of the localized tissue abnormality 82 as somewhere along the geometrically calculated surface. It can be seen from FIG. 9 that the plurality of geometrically calculated surfaces produced by several reflected signal components reduce the possible locations of a localized tissue abnormality. Additionally, the plurality of geometrically calculated surfaces provides information relating to the size of the detected localized tissue abnormality 82.

FIG. 10 is a sectional view of the sensor garment 12 further illustrating the detection of a localized tissue abnormality 82 by direct line-of-flight signal components 96. Considering the detection techniques discussed with FIG. 7, a present localized tissue abnormality 82 is detected by a direct line-of-flight signal component and considered positioned between the two corresponding sensors 18. Therefore, a plurality of direct line-of-flight signal components, encountering a localized tissue abnormality 82 from varying perspectives, is able to reveal a specific location of the tissue abnormality. Thus, in the illustrated embodiment, the intersections of detecting direct line-of-flight signal components 96 reveal the location of the localized tissue abnormality 82. Those skilled in the art will recognize that although FIG. 9 illustrates a two dimensional plane of signal components, the sensor garment 12 provides three dimensional coverage of a breast, thus allowing the sensors 18 to produce a location of a localized tissue abnormality anywhere in the breast.

FIG. 11 is a sectional view of the sensor garment 12 illustrating the planar signal coverage provided by multiple transmitting and receiving sensors 18.

FIGS. 7, 7a, 7b, 9, 10, and 11 illustrate a section of the garment 12 and the illustrated sensors are all located in one plane. Each cup 14, 16 of the garment 12 has numerous sensors 18 located adjacent each other, providing three-dimensional coverage of the breast. For x number of sensors 18, there are theoretically x*(x−1) signal paths available. That is, for a garment cup 14, 16 with 30 sensors, 870 direct signal paths would be generated if each sensor 18 transmitting a signal is received by every other sensor 18. In practice, the number of direct signal paths is less because the sensors 18 adjacent the transmitting sensor 18, which would lie in almost the same plane as the transmitting sensor 18, would not be able to receive a useful signal. This condition is illustrated in FIG. 11. In the illustration, only the direct line-of-flight signal components are depicted in order to maintain intelligibility of the figure. The intersecting signals indicate that any localized tissue abnormality will be encountered numerous times from numerous perspectives.

By considering the reflected signals, the number of signals received is increased over the number of direct signals received. These additional signals are useful for refining the location of any localized tissue abnormality. Additionally, the patient's rib cage and associated musculature will produce a wall of reflected signals indicating the extent of the breast examination. Any abnormalities located adjacent the rib cage would be indicated by reflected signals.

After the signal data is collected and stored for each breast, the raw data is further processed to produce a virtual breast, which is a map of tissue density within a patient's breast. With periodic examinations, a chronological profile of virtual breasts is constructed for a patient. The virtual breasts are compared with regard to time and any long-term changes within the breast tissue are detected. These long-term changes are typically indicators of cancerous tumors. In one embodiment, the raw data is processed using Fourier transforms to reduce the data such that a doctor can perform meaningful diagnoses and analysis.

The remote processing device 22 stores the data collected from each breast examination. The data from each breast examination is added to a chronological profile of the patient's breast tissue development. The chronological profile contains a record of breast examinations over a period of examinations. The chronological profile provides an indication, over time, of tissue density changes in the patient's breasts. These changes may be due to normal changes of the breast tissue, or they may be due to a cancerous growth. The data in the chronological profile is available, in one embodiment, for re-analysis with updated or different software to determine and/or identify changes in the breast tissue over time.

The number of sensors 18 and the size of the transceiver 28 determine the resolution and precision of the cancer detection system 10. In one embodiment, the sensors 18 are positioned within the sensor garment 12 such that they produce a resolution capable of detecting a localized tissue abnormality with a diameter of only a few millimeters.

The features of the present reveal a self-contained cancer detection system capable of reliably detecting an existing localized tissue abnormality in its primary stages of development. Because of the structure and operational behavior of the elements of the cancer detection system 10, a trained ultrasonographer is not required to operate the device. Therefore, the cancer detection system 10 is used and operated by the patient herself in the privacy and convenience of her own home. The privacy and convenience associated with the use of the cancer detection system 10 promote more frequent breast examinations for more women. This, in turn, leads to more early detections of breast cancer, which lead to more successful treatments for this fatal disease.

From the foregoing description, those skilled in the art will recognize that a system for detecting breast tumors offering advantages over the prior art has been provided. The system provides an at-home breast examination that ultrasonically maps the breast tissue of a patient without the requirement of a trained ultrasonographer and relays the results of the examination to the remote processing device by way of the Internet or other transmission media. Additionally, the system builds a chronological profile of the patient's breast tissue structure that can be analyzed automatically or by a physician to monitor abnormal developments in the breast tissue. Finally, the system accounts for user error such as not wearing the garment in the same position for each examination by referencing the discussed chronological profile.

While the present invention has been illustrated by description of several embodiments and while the illustrative embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and methods, and illustrative examples shown and described. Accordingly, departures may be made from such details without departing from the spirit or scope of applicant's general inventive concept.

1. A cancer detection system for mapping breast tissue to detect localized tissue abnormalities and for constructing a chronological profile of a patient's tissue to detect the development of cancerous tumors, said system comprising:
a garment adapted to fit over at least one breast;
a plurality of sensors mounted on said garment, said plurality of sensors including at least one transmitter and a plurality of receivers, each of said plurality of sensors having a surface adapted to be in direct contact with said at least one breast, said plurality of sensors being ultrasonic; and
a processing device in communication with said plurality of sensors, said processing device controlling said at least one transmitter, said processing device acquiring and storing data received from said plurality of receivers.

2. The system of claim 1 wherein said garment is a bra-type garment.

3. The system of claim 1 wherein each of said plurality of sensors include a coupling agent, said coupling agent forming said surface, whereby said coupling agent provides connectivity between said sensors and said at least one breast.

4. The system of claim 1 wherein said plurality of sensors are ultrasonic transceivers.

5. The system of claim 1 wherein said plurality of sensors are piezoelectric.

6. The system of claim 1 wherein said processing device includes a local processing device in communication with a remote processing device, said local processing device acquiring said data and said remote processing device storing and processing said data.

7. The system of claim 1 wherein said processing device processes said data using amplitude analysis and time-of-flight analysis of a signal sent directly from said at least one transmitter to at least one of said plurality of receivers.

8. The system of claim 1 wherein said processing device constructs a chronological profile corresponding to a plurality of breast examinations.

9. The system of claim 9 wherein said processing device references said chronological profile in order to compensate for differences in a position of said garment relative to said at least one breast.

10. A cancer detection system for ultrasonically mapping breast tissue to detect localized tissue abnormalities and for constructing a chronological profile of a patient's tissue to detect the development of cancerous tumors, said system comprising:
a means for positioning a plurality of sensors about a breast;
a means for acquiring data by utilization of said plurality of sensors; and
a means for processing acquired data.

11. The system of claim 10 wherein said means for positioning is a garment worn by the patient.

12. The system of claim 10 wherein said plurality of sensors is a plurality of ultrasonic transceivers.

13. The system of claim 10 wherein said means for processing includes a processor for analysis of a plurality of amplitude and time-of-flight of signals received by said plurality of sensors.

14. The system of claim 10 wherein said means for processing does not include analysis of backscattered signals.

15. The system of claim 10 wherein said means for processing includes a processor for constructing a chronological profile corresponding to a plurality of examinations.

16. A cancer detection system for ultrasonically mapping breast tissue to detect localized tissue abnormalities and for constructing a chronological profile of a patient's tissue to detect the development of cancerous tumors, said system comprising:
a plurality of transmitting sensors adapted to be positioned about a breast;
a plurality of receiving sensors adapted to be positioned about a breast; and
a processing device in electrical communication with said plurality of transmitting sensors and said plurality of receiving sensors, said processing device sensitive to a time-of-flight of a plurality of signals transmitted from said plurality of transmitting sensors and received by said plurality of receiving sensors, said processing device sensitive to an amplitude of said plurality of signals.

17. The system of claim 16 wherein each of said plurality of transmitting sensors is an ultrasonic transmitter.

18. The system of claim 16 wherein each of said plurality of receiving sensors is an ultrasonic receiver.

19. The system of claim 16 wherein each of said plurality of receiving sensors and each of said plurality of transmitting sensors is an ultrasonic transceiver.

20. The system of claim 16 wherein said processing device constructs a chronological profile corresponding to a plurality of examinations.

21. The system of claim 16 wherein said processing device detects the presence of a localized tissue abnormality by analyzing a time-of-flight and an amplitude of said plurality of signals.

22. A cancer detection method for ultrasonically mapping breast tissue to detect localized tissue abnormalities and for constructing a chronological profile of a patient's tissue to detect the development of cancerous tumors, said method comprising the steps of:
a) transmitting an ultrasonic signal from a transmitter through a breast, then;
b) receiving said ultrasonic signal by an array of receivers positioned on an opposite side of said breast relative to said transmitter;
c) analyzing received ultrasonic signal in terms of signal amplitude; and
d) analyzing received ultrasonic signal in terms of signal time-of-flight;
whereby said signal amplitude analysis and said signal time-of-flight analysis indicate the presence of a localized tissue abnormality.

23. The method of claim 22 further including the step of performing a background noise test prior to said transmitting step a).

24. The method of claim 22 further including the step of performing a distance test prior to said receiving step b).

25. The method of claim 24 wherein said distance test includes determining a time-of-flight value such that an initiation and duration of sampling a received signal can be calculated.

26. The method of claim 22 further including the step of constructing a chronological profile corresponding to a plurality of examinations.
